(19) United States
(12) Patent Application Publication
(10) Pub. No.: US 2026/0054100 A1
(43) Pub. Date: Feb. 26, 2026

(54) FOCUSED ULTRASOUND ACUPOINT THERAPY AND HEALTH CARE DEVICE AND METHOD

(71) Applicant: National Health Research Institutes, Miaoli County (TW)

(72) Inventors: Maxim SOLOVCHUK, Miaoli County (TW); Hong-An DENG, Miaoli County (TW)

(21) Appl. No.: 19/306,548

(22) Filed: Aug. 21, 2025

Related U.S. Application Data
(60) Provisional application No. 63/685,397, filed on Aug. 21, 2024.

(51) Int. Cl. A61N 7/00

(52) U.S. Cl. CPC A61N 7/00; A61N 2007/0047; A61N 2007/0073

(57) ABSTRACT

The present invention relates to a focused ultrasound acupoint health-care device comprising an ultrasound output module, a control module, a power supply module, and a human-machine interface module. The device delivers focused ultrasound precisely to a target acupoint, applying mechanical and thermal stimulation, and may automatically adjust output power and treatment duration based on user-specific parameters such as age, weight, height, and acupoint location. The device can be configured as handheld or wearable and may include interchangeable transducers. In certain embodiments, a temperature monitoring function is provided to stop output when the contact area temperature reaches a preset upper limit and resume output when it falls below a preset lower limit, thereby reducing burn risks. The invention is applicable for health care, relaxation, and enhancement of body functions through acupoint stimulation.

### TECHNICAL FIELD OF THE INVENTION

The present invention relates to the field of medical devices and traditional Chinese medicine (TCM) external therapy, and more particularly to a non-invasive focused ultrasound device and method for stimulating human body acupoints, serving as an alternative to traditional moxibustion therapy for treatment and health care.

### BACKGROUND OF THE INVENTION

Moxibustion therapy is one of the external treatment methods in traditional Chinese medicine (TCM), in which moxa floss is ignited and placed near acupoints or affected areas to produce warm stimulation and herbal effects, thereby promoting circulation of qi and blood and improving bodily functions. This method has a long history and is widely used in clinical practice. However, due to the prolonged heating required, there is a significant risk of low-temperature burns, which may cause full-thickness skin damage. If not properly treated, such injuries may lead to infection. Moreover, the efficacy of moxibustion depends heavily on the practitioner's experience and technique, and improper operation can result in inconsistent outcomes.

Traditional ultrasound therapy is commonly used in the field of physical therapy. It employs high-frequency sound waves to penetrate tissue, producing mechanical vibration and thermal effects that promote local blood circulation and tissue repair. Although it is non-invasive and controllable, conventional applications are not specifically designed for TCM acupoints and lack the capability to simultaneously deliver mechanical and concentrated thermal stimulation. In addition, most existing ultrasound devices operate in single-point or single-mode configurations, cannot be optimally adjusted for different user needs, and lack overheat protection mechanisms to prevent burns.

Accordingly, there remains a need for a device that integrates TCM acupoint theory with modern non-invasive energy-based therapy, capable of precisely targeting acupoints, delivering controlled mechanical and thermal stimulation, reducing burn risks, expanding applicability, and ensuring ease of operation with stable performance.

### SUMMARY OF THE INVENTION

The present invention provides a focused ultrasound acupoint health-care device, comprising: an ultrasound output module 1 configured to generate and emit focused ultrasound to a target acupoint so as to apply mechanical and thermal stimulation to the human acupoint; a control module connected to the ultrasound output module 1, configured to set, store, and adjust operating parameters, the parameters including at least one of ultrasound frequency, output intensity, pulse characteristics, duty cycle, and treatment duration; a power supply module configured to provide the energy required for operation of the device; and a human-machine interface module configured to receive user input and display operational status.

In some embodiments, the output frequency range of the ultrasound output module 1 is 0.02 to 10 MHz.

In some embodiments, the device adopts a handheld configuration, a wearable configuration, or a combination thereof.

In some embodiments, the ultrasound output module 1 further comprises a replaceable transducer.

In some embodiments, the transducer is of a flat type or a conical type.

In some embodiments, the control module comprises a pre-stored depth-power correspondence database storing target depth and corresponding power data for different genders, body types, and acupoint locations, and allowing a user to manually select an applicable combination.

In some preferred embodiments, the control module further, based on the database, in combination with the user's age, weight, height, and selected acupoint location, automatically calculates a personalized corrected target depth and corresponding power.

In some embodiments, the device further comprises a temperature monitoring module configured to detect the temperature at a contact area between the ultrasound output module 1 and the user's skin, and to automatically stop output when the temperature reaches a preset upper limit and resume output when the temperature falls below a preset lower limit, thereby preventing burns.

In some preferred embodiments, the preset upper limit is 45° C. and the preset lower limit is 40° C.

In some embodiments, the device is applicable for health care, relaxation, or stimulation to promote body functions through human acupoints.

### BRIEF DESCRIPTION OF THE DRAWINGS

FIG. 1 is an external view illustrating one embodiment of the handheld device of the present invention.

FIG. 2 is an external view illustrating another embodiment of the handheld device of the present invention.

FIG. 3 is an external view illustrating one embodiment of the wearable device of the present invention.

FIG. 4 is a graph showing the relationship between the power and the operating time of the ultrasonic transducer. The curve illustrates the variation in the time required to achieve an equivalent therapeutic effect under different power levels.

FIG. 5 is a graphical representation of the acupoint depth and corresponding power for selected male and female acupoint locations.

### DETAILED DESCRIPTION OF THE INVENTION

The focused ultrasound acupoint health-care device of the present invention utilizes the penetration and energy-focusing characteristics of focused ultrasound to direct acoustic energy to specific acupoints of the human body, thereby producing combined mechanical and thermal stimulation effects. In addition to the thermal effects typically associated with ultrasound, this process induces cavitation phenomena within the tissue fluid and blood vessels, which constitute one of the key technical mechanisms of the invention.

During operation of the focused ultrasound acupoint health-care device, ultrasound energy penetrates the skin and is delivered to the blood vessels and tissue fluid surrounding the target acupoint. The high-frequency alternating pressure field induces cavitation phenomena. The cavitation phenomena can be categorized as follows:

Stable cavitation: Gas nuclei or dissolved gases present in the liquid expand during the low-pressure phase and contract during the high-pressure phase, forming periodically oscillating bubbles. The vibration of these bubbles generates localized microstreaming, which applies subtle mechanical forces to vascular endothelium and surrounding tissues.

Inertial cavitation: Under certain energy conditions, bubbles rapidly collapse during the high-pressure phase, releasing localized instantaneous high temperatures and high-pressure shock waves, thereby further enhancing tissue stimulation effects.

The above mechanical actions can temporarily increase the permeability of local blood vessels and cell membranes, promoting fluid exchange and circulation. At the same time, the localized thermal effects produced by inertial cavitation overlap with the overall thermal effects of focused ultrasound, enhancing the stimulation effect on acupoints.

The invention employs adjustable ultrasound frequency, duty cycle, and output power to control the extent of cavitation, ensuring that the desired health-care and functional stimulation effects are achieved within a comfortable and safe range.

The duty cycle refers to the ratio of “ultrasound emission time” within one cycle and is an important parameter for controlling the strength of ultrasound energy output.

Adjusting the duty cycle allows control over the amount of heat and mechanical stimulation applied to the acupoint, accommodating different users' tolerance levels and health-care needs while reducing the risk of overheating or discomfort.

The preferred embodiments of the present invention will be described in detail below with reference to the accompanying drawings. However, the scope of the present invention is not limited thereto.

### Structural Composition

The focused ultrasound acupoint health-care device of the present invention is composed of multiple functional modules, including:

(1) Ultrasound Output Module 1: Configured to generate and emit focused ultrasound to a target area so as to provide mechanical and thermal stimulation to human acupoints. This module may include one or more focused ultrasound transducer elements and associated support structures.

(2) Control Module: Configured to set, store, and adjust the operating parameters of the device, which may include frequency, intensity, pulse characteristics, and duration of action. This module may be connected to and control the operation of the ultrasound output module 1 via circuitry or a processor.

(3) Power Supply Module: Configured to provide the energy required for operation of the device, and may adopt forms such as mains power, batteries, rechargeable power sources, or other energy supply means. In some embodiments, the power supply module includes an external power/data interface 5 for the transmission of both electrical power and data.

(4) Human-Machine Interface Module: Configured to receive user commands and display the device status, comprising a display screen 2 and control keys 3. The module may employ buttons, knobs, touch panels, display screens, or any combination thereof.

### Control Module

The control module of the present invention is configured to manage and execute the operational settings of the device. Its functions may include pre-storing multiple sets of operational parameter combinations and recording personalized settings for specific users, enabling rapid retrieval and repeated use in subsequent operations. The operational parameters may include output frequency, output intensity, pulse characteristics, duty cycle, cycle time, and other control items related to focal depth and energy output. By appropriately setting the above parameters, the device can deliver corresponding output power according to different usage needs to achieve health-care effects such as relaxation, soothing, or promoting bodily functions.

### Usage Method

The device of the present invention can apply focused ultrasound to selected acupoint locations according to user requirements. Before use, the device may be positioned or fixed at the target location, and an appropriate coupling medium (such as conductive gel) may be applied between the probe and the skin to reduce energy loss and improve transmission efficiency. Once activated, the focused ultrasound can operate according to preset or user-selected modes, with operating time and output power adjustable within a safe range, and with the option of single-point or multi-point simultaneous application. Usage frequency, duration, and intervals can be set according to different user conditions, application purposes, or professional recommendations to achieve the desired health-care and soothing effects.

### Parameter Settings

The device of the present invention may provide multiple adjustable operational parameters to accommodate various usage scenarios and needs. The parameters may include, but are not limited to: ultrasound frequency (covering low to high ranges applicable for medical or health-care purposes, e.g., 0.02-10 MHz); output intensity (adjustable via duty cycle or other energy control methods); pulse characteristics (including pulse length, cycle time, and repetition patterns); single-session duration (configurable within a safe and comfortable range, e.g., 5-30 minutes); and multi-point output functionality (capable of simultaneously acting on multiple target locations). These parameters may be pre-stored in the control system for quick recall when needed, or adjusted in real time according to current requirements, ensuring that the device operates stably and effectively under various application conditions.

### Embodiments

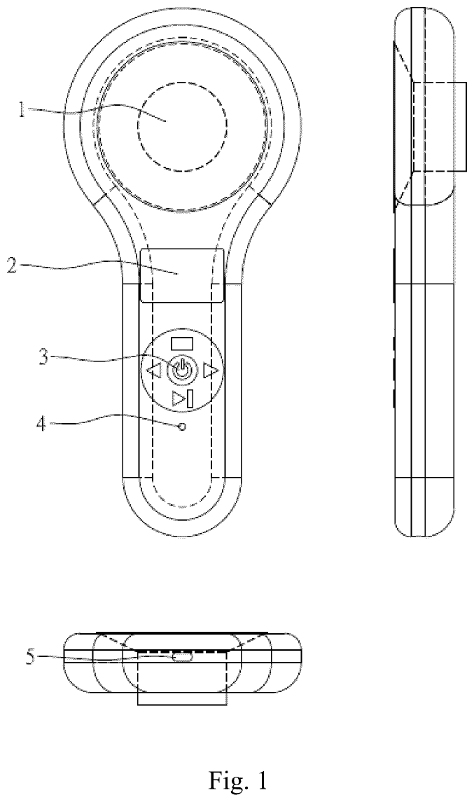

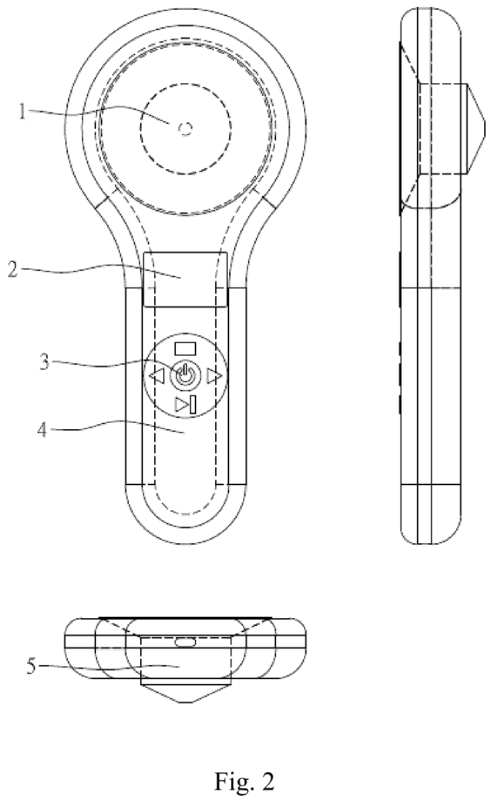

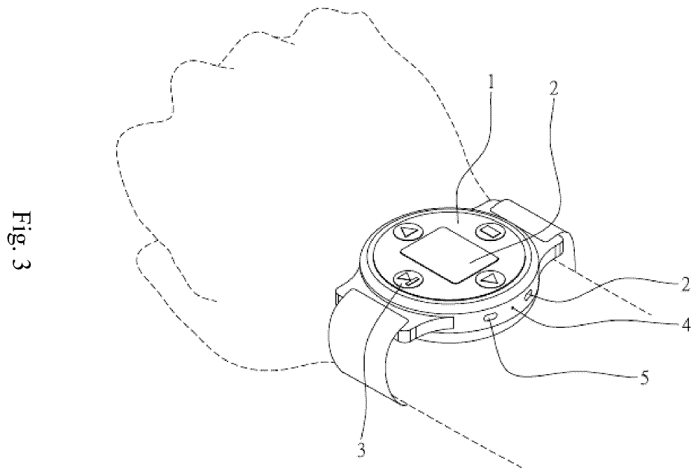

In one specific embodiment, the health-care device of the present invention may be configured in various structural forms, including but not limited to: a handheld structure suitable for operation on a single acupoint (e.g., as shown in FIGS. 1 and 2), and a wearable structure capable of acting on multiple acupoints simultaneously and being secured to a limb (e.g., as shown in FIG. 3), which is provided with connection lugs (6) for securing with cords, straps, or the like to a body part, making it suitable for prolonged wear or multi-point wellness applications. The ultrasonic probe may be manufactured in flat, conical, or other geometric shapes and may be replaceable according to usage requirements.

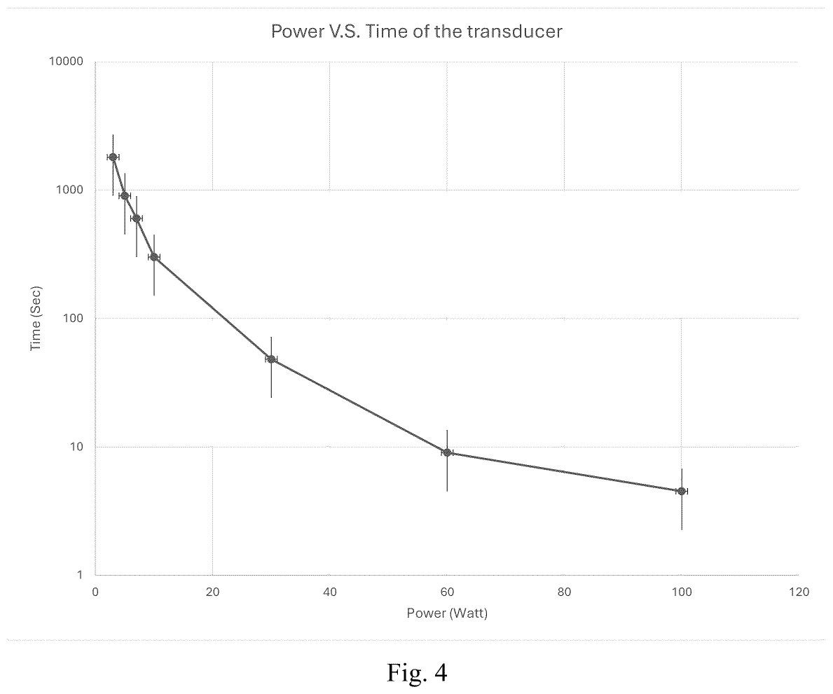

In another embodiment, the device's operation interface may employ buttons, rotary knobs, touch panels, or combinations thereof, allowing convenient adjustment of operating modes and parameters. There exists a specific correlation between the treatment duration and the applied power, as illustrated in FIG. 4. To achieve an equivalent therapeutic effect, a shorter treatment time requires a correspondingly higher output power. For safety considerations, when the required power exceeds 20 W, it is recommended that the procedure be performed by medical personnel with appropriate professional qualifications to minimize the risk of improper use. The device may be equipped with intelligent assistance functions to automatically set parameters for first-time use, or to directly recall previously stored personalized parameter sets. The preferred treatment time is 20 minutes, with a maximum of 30 minutes and a minimum of 5 minutes, and treatment may be paused or resumed during operation.

The device may optionally include temperature display and regulation functions, for example, pausing ultrasonic and heating output when the temperature exceeds a preset upper limit and resuming operation when the temperature returns to the set range. For instance, when the temperature exceeds 45° C., the ultrasonic and heating modules stop operating, with both audible and visual alerts, and when the temperature falls below 40° C., operation resumes. Temperature levels may be set to low, medium, or high, with the heating module's constant temperature set at 30° C. for the medium level, off for the low level, and 35° C. for the high level.

In another embodiment, the device of the present invention may be applied to human acupoint health care according to the following steps:

1. According to usage needs or professional recommendations, select the corresponding acupoint location and position the ultrasonic probe of the device at that location.

2. (Optional) Apply an appropriate coupling medium (such as conductive gel) between the probe surface and the skin to improve energy transmission efficiency.

3. Activate the device to deliver focused ultrasound to the target acupoint to produce the desired mechanical and thermal stimulation.

4. The treatment regimen may be twice daily, 20 minutes per session, with 15 days constituting one treatment cycle, for a total of 1-3 cycles, with a 2-day interval between cycles.

### Verification of Acupoint Depth and Corresponding Power

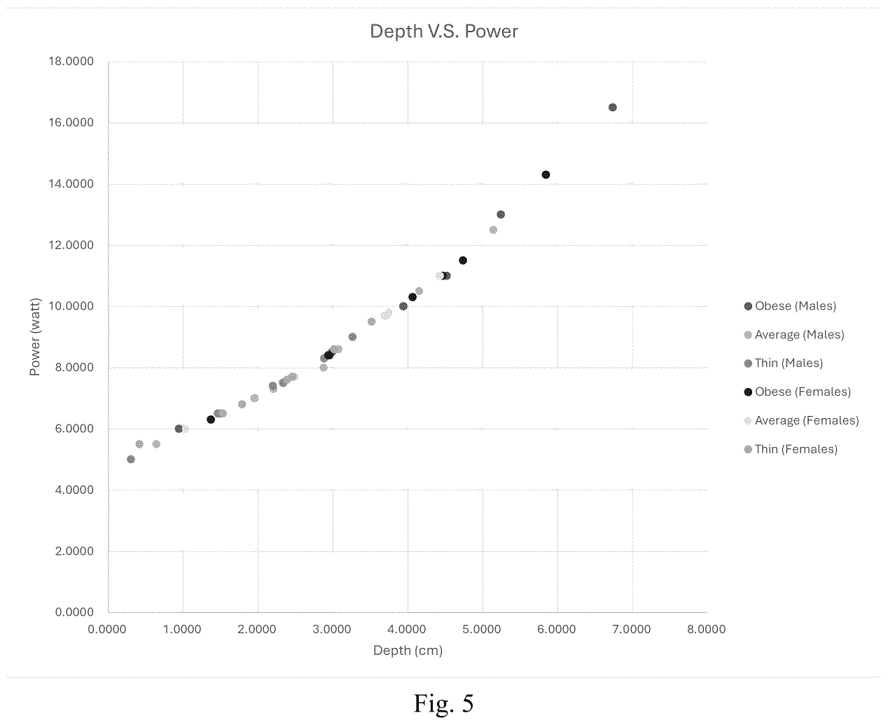

In the application of the present invention, the tissue depth at different acupoint locations affects the transmission efficiency of focused ultrasound energy and the required output power. To establish baseline parameters, tests were conducted for different genders and body types (e.g., obese, standard, and lean) to obtain corresponding data on acupoint depth and required power (Tables 1 and 2, FIG. 5). These data may serve as preset values for system computation, providing an initial reference setting in the absence of personalized data. In some embodiments, the device further includes a reset feature, such as reset pinhole 4, configured to restore the device to its initial factory settings.

TABLE 1
Average Depth and Corresponding Power Requirements
for Selected Acupoint Locations in Males

| | Males | | | | | |
|---|---|---|---|---|---|---|
| | Obese Group | | Average Group | | Thin Group | |
| Acupuncture | | Power | | Power | | Power |
| points | MEAN | (Watt) | MEAN | (Watt) | MEAN | (Watt) |
| Tian Tu | 3.9360 | 10.0000 | 2.8727 | 8.0000 | 2.3350 | 7.5000 |
| (CV-22) | | | | | | |
| Xuan Ji | 0.9400 | 6.0000 | 0.6409 | 5.5000 | 0.3000 | 5.0000 |
| (CV-21) | | | | | | |
| Yu Zhong | 2.9840 | 8.5000 | 2.2046 | 7.3000 | 1.4950 | 6.5000 |
| (KI-26) | | | | | | |
| Bu Lang | 2.3320 | 7.5000 | 1.9500 | 7.0000 | 1.4600 | 6.5000 |
| (KI-22) | | | | | | |
| Qi Hu | 5.2400 | 13.0000 | 4.1500 | 10.5000 | 2.8800 | 8.3000 |
| (ST-13) | | | | | | |
| Yin Men | 6.7320 | 16.5000 | 5.1409 | 12.5000 | 3.2600 | 9.0000 |
| (LU-2) | | | | | | |
| Yuan Ye | 4.5160 | 11.0000 | 3.0727 | 8.6000 | 2.1950 | 7.4000 |
| (GB-22) | | | | | | |

TABLE 2
Average Depth and Corresponding Power Requirements
for Selected Acupoint Locations in Females

| | Females | | | | | |
|---|---|---|---|---|---|---|
| | Obese Group | | Average Group | | Thin Group | |
| Acupuncture | | Power | | Power | | Power |
| points | MEAN | (Watt) | MEAN | (Watt) | MEAN | (Watt) |
| Tian Tu | 4.4600 | 11.0000 | 3.6889 | 9.7000 | 3.0143 | 8.6000 |
| (CV-22) | | | | | | |
| Xuan Ji | 1.3667 | 6.3000 | 1.0333 | 6.0000 | 0.4143 | 10.5000 |
| (CV-21) | | | | | | |
| Yu Zhong | 2.9333 | 8.4000 | 2.4611 | 7.7000 | 1.5286 | 6.5000 |
| (KI-26) | | | | | | |
| Bu Lang | 2.9476 | 8.4000 | 2.4944 | 7.7000 | 1.7857 | 6.8000 |
| (KI-22) | | | | | | |
| Qi Hu | 4.7333 | 11.5000 | 3.7500 | 9.8000 | 2.4571 | 7.7000 |
| (ST-13) | | | | | | |
| Yin Men | 5.8400 | 14.3000 | 4.4222 | 11.0000 | 3.5143 | 9.5000 |
| (LU-2) | | | | | | |
| Yuan Ye | 4.0600 | 10.3000 | 3.7222 | 9.7000 | 2.3857 | 7.6000 |
| (GB-22) | | | | | | |

In practical use, individual factors such as the user's age, weight, and height can affect the actual depth of acupoints. To enhance the precision and adaptability of the output power and focal depth settings, the present invention incorporates the acupoint depth prediction fitting equations shown in Table 3. These equations adjust the preset depth data based on the user's basic input parameters, thereby generating a personalized corrected target depth that more closely reflects the actual condition.

TABLE 3
Fitting Equations for Predicting the Depth of Selected Acupoints

| Acupuncture | | | | | | |
|---|---|---|---|---|---|---|
| points | B0 | B1 | B2 | B3 | R2 | F |
| Tian Tu | −0.4666 | −1.1710 | 0.0075 | 0.0067 | 0.4181 | 18.3211 |
| (CV-22) | | | | | | |
| Xuan Ji | −1.1390 | −0.6100 | 0.0070 | 0.0026 | 0.5546 | 31.7466 |
| (CV-21) | | | | | | |
| Hua Gai | −1.3504 | −0.5413 | 0.0033 | 0.0050 | 0.5558 | 31.9106 |
| (CV-20) | | | | | | |
| Zi Gong | −0.9634 | −0.5905 | 0.0063 | 0.0023 | 0.5723 | 34.1204 |
| (CV-19) | | | | | | |
| Yu Tang | −0.9877 | −0.6959 | 0.0070 | 0.0023 | 0.5896 | 36.6336 |
| (CV-18) | | | | | | |
| Dan Zhong | −1.0358 | −0.8274 | 0.0084 | 0.0029 | 0.6336 | 44.0964 |
| (CV-17) | | | | | | |
| Zhong Ting | −1.6082 | −1.0709 | 0.0147 | 0.0060 | 0.6367 | 44.6807 |
| (CV-16) | | | | | | |

R2: Coefficient of Determination
Y = B0 + B1X1 + B2X2 + B3X3
Y: Predicted Depth of the Acupuncture Point (cm)
X1: Age (Years),
X2: Height (cm),
X3: Weight (Kg)

The control module and intelligent assistance software of the present invention can integrate the corrected target depth with the depth-power correspondence database to calculate the corresponding output power, ultrasonic frequency, duty cycle, pulse parameters, and operating time. The calculated results can be directly applied to device operation or fine-tuned by the user according to personal comfort. During operation, the system can perform dynamic optimization based on real-time feedback signals from sensors, ensuring that energy is accurately delivered to the target depth while enhancing the consistency, repeatability, and overall efficiency of the health-care effect.

While various embodiments of the present invention have been described above, it will be understood that the invention is not limited to the specific configurations disclosed herein. Certain embodiments may provide advantages such as precise targeting of human acupoints, combined mechanical and thermal stimulation, non-invasive operation, customizable parameter settings, multi-acupoint simultaneous operation, and compact form factor suitable for home or portable use.

1. A focused ultrasound acupoint health-care device, comprising:
an ultrasonic output module configured to generate and deliver focused ultrasound to a target acupoint position so as to apply mechanical and thermal stimulation to a human acupoint;
a control module connected to the ultrasonic output module and configured to set, store, and adjust operating parameters, the parameters including at least one of:
ultrasonic frequency, output intensity, pulse characteristics, duty cycle, and operating time;
a power supply module configured to provide the energy required for operation of the device; and
a human-machine interface module configured to receive user input and display the operating status.

2. The device according to item 1, wherein the ultrasonic output module has an output frequency range of 0.02 to 10 MHz.

3. The device according to item 1, wherein the device adopts a handheld structure, a wearable structure, or a combination thereof.

4. The device according to item 1, wherein the ultrasonic output module further comprises a replaceable probe.

5. The device according to item 4, wherein the probe is a flat-type probe or a conical-type probe.

6. The device according to item 1, wherein the control module comprises a pre-stored depth-power correspondence database storing target depths and corresponding power data for different genders, body types, and acupoint positions, and allowing a user to manually select an applicable combination.

7. The device according to item 6, wherein the control module further, based on the database, combines the user's age, weight, height, and selected acupoint position to automatically calculate a personalized corrected target depth and corresponding power.

8. The device according to item 1, further comprising a temperature monitoring module configured to detect the temperature of the contact area between the ultrasonic output module and the user's skin, and to automatically stop output when the temperature reaches a preset upper limit, and resume output when the temperature drops to a preset lower limit, so as to prevent burns.

9. The device according to item 8, wherein the preset upper limit is 45° C. and the preset lower limit is 40° C.

10. The device according to item 1, wherein the device is used for health care, relaxation, or stimulation to promote body functions at human acupoints.
